(19) United States
(12) Patent Application Publication
(10) Pub. No.: US 2015/0290357 A1
(43) Pub. Date: Oct. 15, 2015

(54) ANTIMICROBIAL ADHESIVE

(71) Applicant: GeneJet Biotech, Inc., Taipei City (TW)

(72) Inventor: Fa-Ter CHU, Taipei City (TW)

(21) Appl. No.: 14/682,507

(22) Filed: Apr. 9, 2015

(30) Foreign Application Priority Data
Apr. 10, 2014 (TW) 103113164

(51) Int. Cl. A61L 24/06; A61L 24/00; A61L 24/04; A61L 24/02

(52) U.S. Cl. CPC A61L 24/06; A61L 24/02; A61L 24/0015; A61L 24/043; A61L 2300/404; A61L 2300/104

(57) ABSTRACT

The invention provides an antimicrobial adhesive comprising a plurality of particulates, a plurality of metallic particles and cyanoacrylate monomer. Each of the particulates attached numbers of the metallic particles on the surface to form a mixture having a specific gravity approximating to that of the cyanoacrylate monomer. Those particulates carrying metallic particles on the surface can be suspended well in the cyanoacrylate monomer without aggregation or precipitation while the cyanoacrylate monomer produces no pre-mature polymerization.

### CROSS-REFERENCE TO RELATED APPLICATIONS

This Non-provisional application claims priority under 35 U.S.C. §119(a) on patent application Ser. No(s). 103113164 filed in Taiwan, Republic of China Apr.10, 2014, the entire contents of which are hereby incorporated by reference.

### FIELD OF THE INVENTION

The present invention is related to an antimicrobial adhesive in the art of medical care, and particularly involves an adhesive prepared by cyanoacrylate monomer.

### BACKGROUND OF THE INVENTION

Cyanoacrylate is one kind of the adhesives, which is often used in industry and household for bonding plastic, rubber, metal, glass and woods etc. Common cyanoacrylate adhesives are methyl-2-cyanoacrylate, ethyl-2-cyanoacrylate, n-butyl cyanoacrylate and 2-octyl cyanoacrylate.

Also, cyanoacrylate is often applied in the medical care as one of the compositions in a tissue adhesive, and is more commonly used for hemostasis and wound closure etc. Conventional methods for wound closure generally utilize sutures, surgery staples or tapes, but sutures may induce foreign reaction so that the sutures have to be taken off after the wound is healed. However, a tissue adhesive is free from the above-mentioned drawbacks and risks, and thus, it is extensively used for medical care.

When cyanoacrylate is applied in the field of medical care, it is desired to have an antimicrobial effect for preventing the infection by germs such as staphylococcus aureus and escherichia coli. While cyanoacrylate has some antimicrobial property, the mechanism of its antimicrobial effect is still unknown. A possible reason is that a high density of the negative charges can be generated after the polymerization of cyanoacrylate monomers, and they are allowed to react with the positive charges on the cell walls of the germs. Therefore, the antimicrobial effect is obtained. However, the antimicrobial effect of cyanoacrylate can be further enhanced by additional antimicrobial agents in cyanoacrylate.

The antimicrobial agents may be classified into three major categories: natural antimicrobials, organic antimicrobials and inorganic antimicrobials. Natural antimicrobials, such as green mustard essential oil, mustard extracts, hinokitiol and chitin, etc., come from the extracted substances from plants. Bounded by the processing conditions of the raw materials, the massive production of natural antimicrobial agents is difficult, and they have disadvantages of short antimicrobial period, poor resistance to heat and poor chemical stability.

Organic antimicrobials include germicides (ethanol, quaternary ammonium salts, triclosan, etc.), preservatives (formaldehyde, organic halogen compound etc.), and anti-fungus agents (pyridine, haloalkane etc.). Organic antimicrobials have advantages of high potency with wide range of antimicrobial effects, but they also have issues such as poor heat resistance, migration, leakage of toxicant from the carrier, poor resistance to washing and short period of effectiveness. Therefore, their usage is greatly limited.

Inorganic antimicrobials mainly utilize the antimicrobial capability of metal such as silver (Ag), copper (Cu), zinc (Zn) and titanium (Ti) etc. By means of physical absorption, ion exchange or multi-layer cladding etc., antimicrobial metal (or its ion) such as Ag, Cu and Ti etc. is incorporated in a layered or porous material such as the zeolite, silica gel and phosphate to enable antimicrobial property in them. Afterwards, antimicrobial agent is added into the corresponding products for its antimicrobial capability. The features of inorganic antimicrobial agents are improved safety, heat resistance, durability and chemical stability. Inorganic antimicrobials are currently used more often in fiber, plastic and building materials than other kinds of antimicrobial agents. However, inorganic antimicrobial agents have poor compatibility with macromolecular materials and easily aggregate in a resin matrix, which brings significant problems to processing such as spinning and film forming etc. and affects the appearance of the product as well.

Regarding the antimicrobial property of silver, the silver content usually indicates the antimicrobial capability of the composition. In addition to the amount of silver ions, the antimicrobial effect is even more related to the valence state that silver ions exist in the carrier. The antimicrobial effect of silver ion is progressively decreased in the following order: Ag³⁺>Ag²⁺>Ag⁺. The silver ion with higher valence state has a higher reduction potential such that it may react with oxygen and generate atomic oxygen with a high antimicrobial property. Therefore, the silver ion with higher valence state may possess a higher antimicrobial effect. However, it is more difficult to make this ion, and the resulting ion has a poor stability. Depending on the carrier, the antimicrobial silver can be classified as ion-exchange-type(ex: silver-zeolite, silver-copper phosphate), absorption-type (ex: silver-silica gel, silver-activated carbon, silver-calcium phosphate)and glass-type(ex: silver-leachable glass), wherein the ion-exchange-type has the strongest antimicrobial capability.

Due to the high reactivity of cyanoacrylate monomers, adding antimicrobial agents such as quaternary ammonium salts etc. may cause premature polymerization of cyanoacrylate monomers, resulting in a reduction in shelf life. Further, this may also impact the polymerization of cyanoacrylate monomers, causing the decrease in the mechanical strength of the polymer and its ability to treat wound.

As for inorganic antimicrobial agents, i.e., metal such as Cu, Ag, Ti or Zn, although they may not cause premature polymerization of cyanoacrylate monomers or impact the polymerization of cyanoacrylate monomers, but the difference in the specific gravity may cause the precipitation of the inorganic antimicrobial agents and the loss of their antimicrobial property.

As a result, an antimicrobial adhesive is provided in the present invention, wherein the added inorganic antimicrobial agent neither causes the premature polymerization of cyanoacrylate monomers nor impacts the polymerization of cyanoacrylate monomers, and furthermore, it may not cause the precipitation over a long period of time.

### SUMMARY OF THE INVENTION

In order to resolve the foregoing problems, the present invention is to provide an antimicrobial adhesive comprising a plurality of particulates, a plurality of metallic particles and a cyanoacrylate monomer, wherein the plurality of particulates may be macromolecular particulates or inorganic particulates. The macromolecular particulates may be one or a mixture of more than one of polypropylene, polyethylene, polyurethane, polyacrylate, polystyrene and polysiloxane, wherein the more than one macromolecular substances may be made into macromolecular particulates by a method of blending, etc. The inorganic particulates may be inorganic pigments, silicon dioxide, aluminum oxide or pulverized coal ash etc. The plurality of metallic particles described in this invention may be a plurality of pure metallic particles, a plurality of metallic oxide particles or a plurality of metallic salts particles, wherein the plurality of pure metallic particles may be one or a combination of more than one of pure metal such as Ag, Cu, Au, Zn, Pt, Pd, Ti, Ir, Zr, Fe, Ru, Mo, Rh and Sn; the plurality of metallic oxide particles may be one or a combination of more than one of metallic oxides such as silver oxide, copper oxide, aluminum oxide, zinc oxide, titanium dioxide, tin oxide and zirconium dioxide; the plurality of metallic salts particles may be one or a combination of more than one metallic salts such as silver nitrate, zinc chloride, chlorauric acid, chlorine molybdate, ruthenium chloride, palladous chloride, rhodium chloride, iron chloride, copper nitrite and palladium acetate. Additionally, the particle size of the metallic particles is less than that of the particulates.

In one embodiment of this invention, the cyanoacrylate monomer has a general formula (I): CH₂C(CN)COOR, wherein R may be one of ethyl, 2-octyl, n-octyl, 2-ethyl hexyl, butyl, dodecyl, methyl, 3-methoxybutyl, 2-butoxyethyl, 2-isopropoxyethyl and 1-methoxy-2-propyl.

In another embodiment of this invention, the present invention is to provide a method for the preparation of an antimicrobial adhesive, including the following steps: (1) mixing a plurality of particulates and a plurality of metallic particles by mechanical coalescences to obtain a mixture, wherein the plurality of particulates may be macromolecular or inorganic particulates, and the particle size of the metallic particles is less than that of the particulates, and the resulting mixture is the particulates with the plurality of metallic particles attached on the surface. In addition, the particulates with the attached plurality of metallic particles may be further compressed to smaller particulates sizes by the way of mechanical coalescences and the particulates with a uniform particle size may be obtained by filtering; (2) flushing the mixture by deionized water; (3) drying the mixture in a vacuum oven; (4) dispersing the mixture in the cyanoacrylate monomer, wherein the specific gravity of the mixture approximates to that of the cyanoacrylate monomer.

The antimicrobial adhesive prepared by the method according to the present invention comprises particulates with a plurality of metallic particles attached, and the specific gravity of this mixture approximates to that of the cyanoacrylate monomer, such that the particulates with a plurality of metallic particles attached may uniformly suspend in the cyanoacrylate monomer without aggregation or precipitation. Furthermore, no premature polymerization may happen to the cyanoacrylate monomer. Therefore, the antimicrobial adhesive of the present invention not only resolves the problem of precipitation from inorganic antimicrobials, but also has great antimicrobial property.

The details of one or more embodiments of the invention are set forth in the description below. Other features or advantages of the present invention will be apparent from the following detailed description of an example and also from the claims.

### BRIEF DESCRIPTION OF THE DRAWINGS

FIG. 1 is the schematic illustration of particulates with a plurality of metallic particles attached, which are included in an antimicrobial adhesive of the present invention.

FIG. 2 is a cross-sectional view that the particulates with a plurality of metallic particles attached, which are included in an antimicrobial adhesive of the present invention, are evenly dispersed on the surface of an object.

FIG. 3 is a cross-sectional view that the particulates with a plurality of metallic particles attached, which are included in an antimicrobial adhesive of the present invention, are randomly dispersed on the surface of an object.

FIG. 4 is a flowchart of the preparation method for an antimicrobial adhesive of the present invention.

### DETAILED DESCRIPTION OF THE INVENTION

The purpose of the antimicrobial adhesive and the principle of adhesion of the present invention have been comprehended by those having ordinary skill in the related art. Thus, in the following description, a detailed description is made only for the specific functions of each composition in the antimicrobial adhesive of the present invention. Additionally, the drawings in the following description may not be depicted according to the real size either. Their functions lie in presenting the schematic illustration related to the characteristics of the present invention.

The terminology “mechanical coalescence” described in the present invention refers to a process performed in a reactor for mechanical coalescence. The reactor for mechanical coalescence comprises a cylindrical chamber with compression tools and blades that is rotating at high speed. The rotating speed is typically higher than 1000 rpm. The plurality of metallic particles 2 and the plurality of particulates 1 described in the present invention are added into the cylindrical chamber. While the cylindrical chamber is rotating, the plurality of metallic particles 2 and the plurality of particulates 1 are squeezed against the wall of the cylindrical chamber. The compression tools and the centrifugal force generated by the high rotating speed promote the coalescence between the plurality of metallic particles 2 and the plurality of particulates 1.

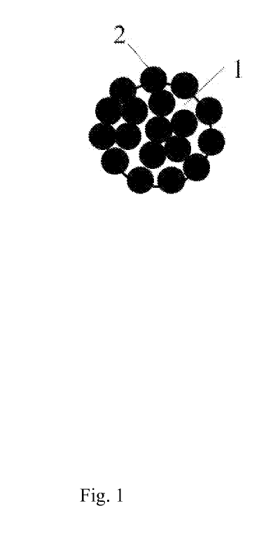

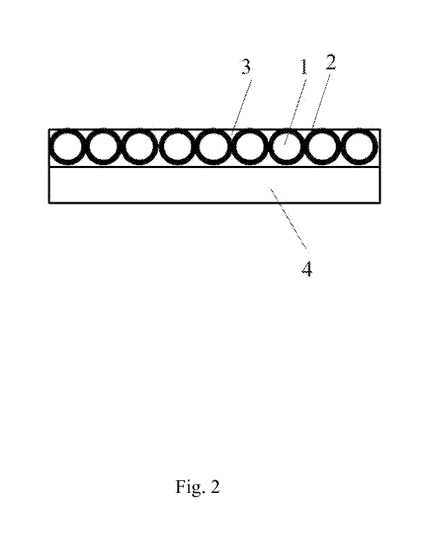

Referring to FIGS. 1 and 2, the present invention is to provide an antimicrobial adhesive comprising a plurality of particulates 1, a plurality of metallic particles 2 and a cyanoacrylate monomer 3, wherein the plurality of particulates 1 may be macromolecular particulates or inorganic particulates. In one embodiment of this invention, the particulates 1 are macromolecular particulates, each of the macromolecular particulates is composed of one or a mixture of more than one material. Typical macromolecular particulates comprise polypropylene, polyethylene, polyurethane, polyacrylate, polystyrene and polysiloxane, wherein the more than one macromolecular substance may be made into macromolecular particulates by blending etc. In another embodiment of this invention, the particulates 1 are inorganic particulates, wherein each of the inorganic particulates is composed of inorganic pigments, silicon dioxide, aluminum oxide or pulverized coal ash. The plurality of metallic particles 2 described in the present invention comprise pure metallic particles, metallic oxide and metallic salts or a mixture of them, wherein the plurality of pure metallic particles are compose of Ag, Cu, Au, Zn, Pt, Pd, Ti, Ir, Zr, Fe, Ru, Mo, Rh and Sn or their mixture; the plurality of metallic oxide particles are composed of silver oxide, copper oxide, aluminum oxide, zinc oxide, titanium dioxide, tin oxide and zirconium dioxide or their mixture; the plurality of metallic salts particles are compose of silver nitrate, zinc chloride, chlorauric acid, chlomolybdic acid, ruthenium chloride, palladous chloride, rhodium chloride, iron chloride, copper nitrite and palladium(II) acetate or their mixture.

For example, the plurality of pure metallic particles 2 attached on the particulate 1 is composed of a plurality of silver particles and a plurality of gold particles, or of a plurality of silver particles, gold particles and platinum particles or their mixture. Similarly, a plurality of metallic oxide or the metallic salts particles attached on the particulate 1 may be composed of metallic oxide or metallic salts or their mixture. In another embodiment of this invention, the plurality of metallic particles 2 attached on the particulate 1 may be composed of a combination of a plurality of pure metallic particles, a plurality of metallic oxide particles and a plurality of metallic salts particles. For example, there are a plurality of pure metallic particles and a plurality of metallic oxides particles on the surface of the particulate 1, or there are a plurality of pure metallic particles, a plurality of metallic oxides particles and a plurality of metallic salts particles on the surface of the particulate 1. One or a combination of more than one thereof may be selected. The particle size of the particulate 1 may be from 10 nm to 100 um and that of the metallic particle 2 may be from 1 nm to 10 um. In yet another embodiment of this invention, each of the particulates 1 with a plurality of metallic particles 2 attached is a macromolecular particulate, the particle size of the macromolecular particulates is from 12 nm to 120 um. The particle size of the metallic particles 2 is less than that of the particulates 1, and the ratio that the particle size of the particulate 1 to that of the metallic particle 2 is from 1.1 to 100000. The preferred ratio of the particle size is from 2 to 1000.

In one embodiment of this invention, the cyanoacrylate monomer 3 has a formula (I): CH₂C(CN)COOR. The structural of the formula (1) is shown as the following,

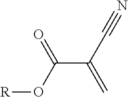

wherein R may be one of ethyl, 2-octyl, n-octyl, 2-ethyl hexyl, butyl, dodecyl, methyl, 3-methoxybutyl, 2-butoxyethyl, 2-isopropoxyethyl and 1-methoxy-2-propyl.

During the fabrication of the antimicrobial adhesive of the present invention, a method of mechanical coalescence is employed to have a plurality of metallic particles 2 attached on the surface of each of the particulates 1. Furthermore, the specific gravity of the particulate 1 with a plurality of metallic particles 2 attached approximates to that of the cyanoacrylate monomer 3, such that the particulate 1 with a plurality of metallic particles 2 attached may uniformly suspend in the cyanoacrylate monomer 3 without aggregation or precipitation. Furthermore, no premature polymerization may happen to the cyanoacrylate monomer 3. In an embodiment, the average of the specific gravity of the particulate 1 with a plurality of metallic particles 2 attached may range from 0.9 to 1.1 g/cm³.

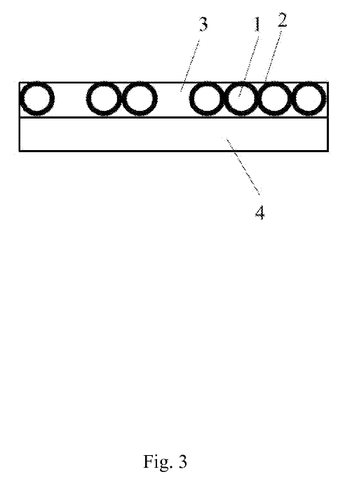

As shown in FIG. 1, FIG. 2 and FIG. 3, Example one of the present invention is to provide an antimicrobial adhesive comprising a plurality of particulates 1, a plurality of metallic particles 2 and a cyanoacrylate monomer 3, wherein the plurality of the particulates 1 are solid or hollow macromolecular particulates, and the weight ratio between the plurality of the metallic particles 2 and the plurality of the macromolecular particulates is 1:9, 2:8, 3:7 or 4:6, wherein the preferred weight ratio is 2:8. In one embodiment of the present invention, the plurality of metallic particles 2 (as shown in FIG. 1) are attached on the surface of each of the macromolecular particulates 1, and the specific gravity of the macromolecular particulate with the plurality of metallic particles 2 attached is approximate to that of the cyanoacrylate monomer 3. By adding various weight ratios between metallic particles 2 and the macromolecular particulates 1 during the coalescence process, the specific gravity of the macromolecular particulate with the plurality of metallic particles 2 attached can be controlled. , The average specific gravity of the macromolecular particulate with the plurality of metallic particles 2 attached ranges from 0.9 to 1.1 g/cm³.

In one embodiment of the present invention, the macromolecular particulate has particle size range from 10 nm to 100 um, and its material is selected from polypropylene, polyethylene, polyurethane, polyacrylate, polystyrene, polysiloxane or their mixture, wherein the mixture may be made into macromolecular particulates by blending etc. The preferred macromolecular particulate is polypropylene. In another embodiment of the present invention, the metallic particle 2 in embodiment 1 has particle size range from 1 nm to 10 um, and is composed of one or more than one of the metallic particles such as Ag, Cu, Au, Zn, Pt, Pd, Ti, Ir, Zr, Fe, Ru, Mo, Rh and Sn. The preferred metallic particle 2 is silver. In yet another embodiment of the present invention, the particle size of the metallic particle 2 is less than that of the macromolecular particulate 1. The plurality of the metallic particles 2 cover the entire surface of each of the macromolecular particulates allowing each of the macromolecular particulates with the attached plurality of the metallic particles 2 to suspend in the cyanoacrylate monomer 3 without aggregation or precipitation. In further another embodiment of the present invention, the plurality of the metallic particles 2 may not be necessarily cover the entire surface of each of the macromolecular particulates, but the macromolecular particulates with the attached plurality of the metallic particles 2 may still uniformly suspend in the cyanoacrylate monomer 3.

In one embodiment of the present invention, the cyanoacrylate monomer 3 has a general formula (I): CH₂C(CN)COOR, where R is selected from ethyl, 2-octyl, n-octyl, 2-ethyl hexyl, butyl, dodecyl, methyl, 3-methoxybutyl, 2-butoxyethyl, 2-isopropoxyethyl and 1-methoxy-2-propyl etc. Depending on the R group, the polymerization time for the cyanoacrylate monomer 3 may change; therefore, the polymerization time may be controlled by adding one or more than One kind of the cyanoacrylate monomers 3 for the requirements of various applications, wherein the polymerization time may be controlled between 30 seconds and 10 minutes as desired. In one embodiment, R group may be 2-octyl, and the polymerization time of 2-octyl-cyanoacrylate may range from 3 to 5 minutes.

As shown in FIG. 2 and FIG. 3, the antimicrobial adhesive prepared by a plurality of macromolecular particulates 1, a plurality of metallic particles 2 and a cyanoacrylate monomer 3 may be used to coat an object 4, and the particle size of the metallic particles 2 is less than that of the macromolecular particulates 1. The object 4 may be biological or non-biological. The biological objects include human bodies or animal skins etc., and the non-biological objects include substances such as medical devices, implant, metallic substrates, macromolecular substrates, glass etc. Furthermore, the surface of the object 4 may be smooth or rough. In one embodiment of the present invention, the surface of the object 4 may be a hard surface or a soft surface. In another embodiment of the present invention, the macromolecular particulates may have a particle size of 10 nm˜100 um and the macromolecular particulates with the plurality of metallic particles 2 attached may have a particle size of 12 nm˜120 um. Additionally, good antimicrobial effect may be obtained when the thickness of the antimicrobial adhesive is 10 nm˜500 um. In a preferred embodiment, the thickness of the antimicrobial adhesive equals to the particle size of the macromolecular particulates with attached plurality of metallic particles 2 (as shown in FIG. 2). In addition, each of the macromolecular particulates with attached plurality of metallic particles 2 may be arranged with even spacing between them (as shown in FIG. 2). As a result, good antimicrobial effect of the antimicrobial adhesive may be obtained with the least amount of the antimicrobial metallic particles 2. In another embodiment, each of the macromolecular particulates with attached plurality of metallic particles 2 may be randomly arranged on the surface (as shown in FIG. 3). In yet another embodiment, the particle size of the metallic particles 2 is 2 nm, the particle size of the macromolecular particulates is 10 nm, and the thickness of the antimicrobial adhesive 3 is 10 nm, the macromolecular particulates are evenly distributed, and the plurality of metallic particles 2 cover the whole surface of the macromolecular particulates, then the effective antimicrobial surface increases by a factor of 11.5 compared to that of the macromolecular particulates without any attached metallic particles 2.

As shown in FIG. 1 to FIG. 3, the Example two of the present invention is to provide another antimicrobial adhesive comprising a plurality of particulates 1, a plurality of metallic particles 2 and a cyanoacrylate monomer 3, wherein the plurality of particulates 1 comprise solid or hollow inorganic particulates. The examples are: inorganic pigments, silicon dioxide, aluminum oxide and pulverized coal ash. During the fabrication of the antimicrobial adhesive in Example two, mechanical coalescence is employed to attach a plurality of metallic particles 2 on the surface of the inorganic particulates. Furthermore, the specific gravity of the inorganic particulates with the attached plurality of the metallic particles 2 is approximate to that of the cyanoacrylate monomer 3, such that the inorganic particulates with attached plurality of metallic particles 2 may suspend in the cyanoacrylate monomer 3 without aggregation or precipitation. Furthermore, no pre-mature polymerization may happen to the cyanoacrylate monomer 3. In Example two, the average specific gravity of the inorganic particulates with attached plurality of metallic particles 2 may range from 0.9 to 1.1 g/cm³. The difference between Example 2 and Example 1 lies in that the particulates 1 in Example 2 are inorganic particulates, and the antimicrobial adhesive described in the Example 2 may be implemented referring to the description in the Example 1.

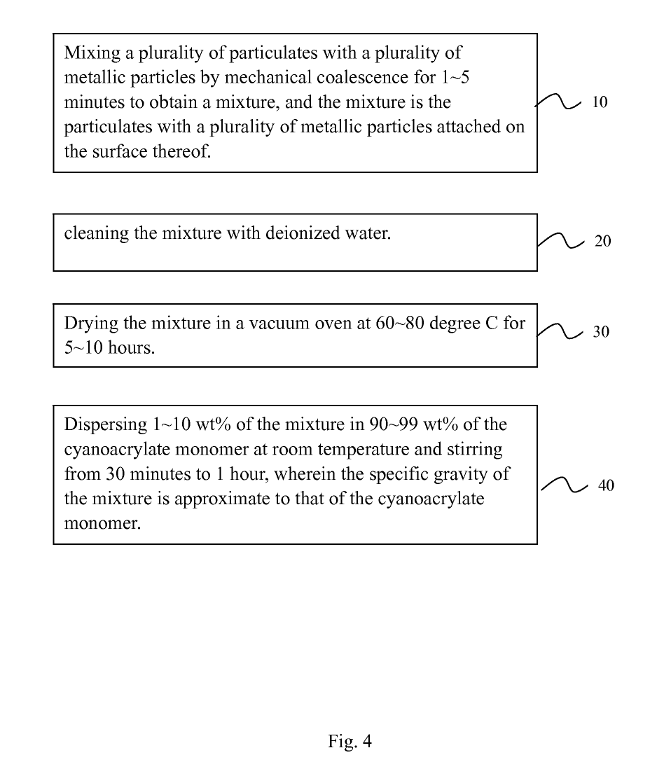

As shown in FIG. 4, the Example 3 of the present invention is to provide a method to prepare an antimicrobial adhesive, which includes the following steps:

Step 10: mixing a plurality of particulates 1 and a plurality of metallic particles 2 by mechanical coalescence for 1˜5 minutes to obtain a mixture, wherein the plurality of the particulates 1 may be macromolecular particulates or inorganic particulates, and the particle size of the metallic particles 2 is less than that of the particulates 1, and the mixture comprises the particulates 1 with attached plurality of metallic particles 2; in addition, the particulates 1 with attached plurality of metallic particles 2 may be further compressed to a smaller particle size after mechanical coalescence and the particulates with an uniform particle size can be obtained by filtering. Step 20: flushing the mixture with deionized water. Step 30: drying the mixture in vacuum at 60˜80 celsius degree for 5˜10 hours. Step 40: dispersing 1˜10 wt % of the mixture in 90˜99 wt % of the cyanoacrylate monomer 3 at room temperature and stirring for 30 minutes˜1 hour, wherein the specific gravity of the mixture approximates to that of the cyanoacrylate monomer 3. In one embodiment, the average specific gravity of the particulates 1 attached with a plurality of metallic particles 2 ranges from 0.9 to 1.1 g/cm³. Additionally, the particulates 1, the metallic particles 2 and the cyanoacrylate monomer 3 described in the Example 3 may be implemented referring to the conditions described in Example 1 and 2 with various particulate contents, metallic particles and coating thickness, etc. According to the preparation method for the antimicrobial adhesive in the Example 3, mechanical coalescence is employed to prepare the polypropylene particulates attached with a plurality of silver particles (an Ag/PP mixture), wherein the weight ratio of the silver particles to the polypropylene particulates may be 2: 8. Further, the particle size of the silver particles may be 0.5˜1 μm with a specific gravity of 3 g/cm³, and the particle size of the polypropylene particulates may be 10˜15 μm with a specific gravity of 0.905 g/cm³. Subsequently, the Ag/PP mixture is dispersed in the 2-octyl cyanoacrylate monomer at room temperature, wherein the specific gravity of 2-octyl cyanoacrylate monomer is 1.04 g/cm³.

The antimicrobial adhesive prepared by the foregoing method is placed in a 7 ml glass vial and it is used to perform stability test for Ag/PP particulates, polymerization test for the cyanoacrylate monomers (as shown in Table 1), antimicrobial test for staphylococcus aureus (as shown in Table 2) and antimicrobial test for escherichia coli (as shown in Table 3).

TABLE 1
Stability test for Ag/PP particulates and polymerization test for
cyanoacrylate monomers.

| | Viscosity | | | |
|---|---|---|---|---|
| | before | Viscosity | | |
| | aging test | after aging | Premature- | |
| | (cP) | test (cP) | polymerization | Precipitation |
| Control | 7 | 10 | — | — |
| (no Ag/PP | | | | |
| particulates) | | | | |
| Sample A | 8 | 14 | None | None |
| Sample B | 8 | 18 | None | None |

TABLE 2
Antimicrobial inhibition zone tests for staphylococcus aureus

| | Diameter of | Increased in the diameter of |
|---|---|---|
| | inhibition | inhibition zone (%) |
| Sample | zone (cm) | (compared to Control) |
| Control (filter paper) | 0.80 | — |
| Experiment C | 0.93 | 16.4 |
| (filter paper + | | |
| 2-octyl cyanoacrylate) | | |
| Experiment D | 1.08 | 35.4 |
| (filter paper + Ag/PP | | |
| Mixture + | | |
| 2-octyl cyanoacrylate) | | |

TABLE 3
Antimicrobial testing for Escherichia Coli

| | Bactericidal activity |
|---|---|
| Sample | (%) |
| Control | 0 |
| Experiment E(Ag/PP mixture + | >99.99 |
| 2-Octyl Cyanoacrylate) | |

In the experiment, sample A in Table 1 underwent an aging test for 2 hours at 120° C., and sample B underwent an aging test for 6 hours at 120° C. The results show that no significant change in the viscosities compared with the Control sample before and after aging test for both sample A and B; furthermore, no precipitation occurred in neither sample A nor sample B, and no pre-mature polymerization happened to 2-octyl cyanoacrylate monomers, either. Therefore, it is concluded that Ag/PP particulates are well dispersed in 2-Octyl Cyanoacrylate monomers without precipitation or causing pre-mature polymerization to 2-octyl cyanoacrylate monomers.

Table 2 shows the results of antimicrobial inhibition zone test based on ATCC6538p. The results show that the increase in the diameters of the antimicrobial inhibition zones are 16.4% and 35.4% compared to the Control in Experiments C and D, respectively. This indicates that the antimicrobial adhesive of the present invention has significant antimicrobial effect against staphylococcus aureus.

Table 3 shows the antimicrobial test results based on JIS Z2801 method. The results show that, compared to the Control, the antimicrobial capability of 2-Octyl Cyanoacrylate (with Ag/PP mixture) is more than 99.99% in experiment E. This indicates the antimicrobial adhesive of the present invention has good antimicrobial effect against Escherichia Coli.

In summary, the antimicrobial adhesive of the present invention comprises the particulates with attached plurality of metallic particles. The specific gravity of those particulates is approximate to that of the cyanoacrylate monomers, so that the particulates with attached plurality of metallic particles may suspend in the cyanoacrylate monomers without aggregation or precipitation. Further, no pre-mature polymerization may happen to the cyanoacrylate monomers. Therefore, the antimicrobial adhesive of the present invention not only resolves the precipitation issue for inorganic antimicrobial agents, but further possesses good antimicrobial property.

While the present invention has disclosed the preferred embodiments in the foregoing description, those preferred embodiments are not intended to limit the scope of the present invention. Modification may be made without departing from the scope of the present invention by those who are skilled in the art. Thus, the scope of the present invention should base on the claims which are appended to this specification.

What is claimed is:

1. An antimicrobial adhesive, comprising:
a plurality of metallic particles;
a plurality of particulates; and
a cyanoacrylate monomer;
wherein the plurality of metallic particles are attached on the surface of each of the plurality of particulates, and a specific gravity of the plurality of particulates with the attached plurality of metallic particles is approximate to a specific gravity of the cyanoacrylate monomer.

2. The antimicrobial adhesive of claim 1, wherein the plurality of metallic particles is selected from a group consisting of a plurality of pure metallic particles, a plurality of metallic oxide particles, a plurality of metallic salt particles and a combination thereof.

3. The antimicrobial adhesive of claim 2, wherein the plurality of pure metallic particles is selected from a group consisting of Ag, Cu, Au, Zn, Pt, Pd, Ti, Ir, Zr, Fe, Ru, Mo, Rh, Sn , and a combination thereof.

4. The antimicrobial adhesive of claim 2, wherein the plurality of metallic oxide particles is selected from a group consisting of silver oxide, copper oxide, aluminum oxide, zinc oxide, titanium dioxide, tin oxide, zirconium dioxide the metallic oxide or their mixture; wherein the plurality of metallic salts particles comprise silver nitrate, zinc chloride, chlorauric acid, chlomolybdic acid, ruthenium chloride, palladous chloride, rhodium chloride, iron chloride, copper nitrite, palladium(II) acetate , and a combination thereof.

5. The antimicrobial adhesive of claim 1, wherein the plurality of metallic particles have a particle size range from 1 nm to 10 μm.

6. The antimicrobial adhesive of claim 1, wherein the plurality of particulates comprise macromolecular particulates.

7. The antimicrobial adhesive of claim 6, wherein the macromolecular particulates is selected from a group consisting of polypropylene, polyethylene, polyurethane, polyacrylate, polystyrene, polysiloxane , and a combination thereof.

8. The antimicrobial adhesive of claim 6, wherein the plurality of macromolecular particulates have a particle size of 10 nm to 100 μm.

9. The antimicrobial adhesive of claim 1, wherein the plurality of particulates comprise inorganic particulates.

10. The antimicrobial adhesive of claim 9,wherein the inorganic particulates is selected from a group consisting of inorganic pigments, silicon dioxide, aluminum oxide and pulverized coal ash , and a combination thereof.

11. The antimicrobial adhesive of claim 1, wherein the specific gravity of the plurality of particulate ranges from 0.9 to 1.1 g/cm³.

12. The antimicrobial adhesive of claim 1, wherein the cyanoacrylate monomer has a general formula of CH₂C(CN)COOR, wherein R is selected from a group consisting of ethyl, 2-octyl, n-octyl, 2-ethyl hexyl, butyl, dodecyl, methyl, 3-methoxybutyl, 2-butoxyethyl, 2-isopropoxyethyl and 1-methoxy-2-propyl.

13. A preparation method for an antimicrobial adhesive, comprising the following steps:
mixing a plurality of particulates with a plurality of metallic particles by mechanical coalescence to obtain a mixture, wherein the mixture comprises the plurality of particulates with the plurality of metallic particles attached on the surface of the plurality of particulates, wherein the particle size of the plurality of metallic particles are less than the particle size of the plurality of particulates;
cleaning the mixture with deionized water;
drying the mixture in an oven;
dispersing the mixture in a cyanoacrylate monomer, wherein a specific gravity of the mixture is approximate to a specific gravity of the cyanoacrylate monomer.

14. The preparation method for an antimicrobial adhesive of claim 13, wherein the specific gravity of the mixture ranges from 0.9 to 1.1 g/cm³.
